(19) United States
(12) Patent Application Publication
(10) Pub. No.: US 2026/0047899 A1
(43) Pub. Date: Feb. 19, 2026

(54) WORKING CHANNEL TOOL ASSEMBLY

(71) Applicant: AURIS HEALTH, INC., Santa Clara, CA (US)

(72) Inventors: Grace Magdalena RESTLE, San Francisco, CA (US); Adrian Tyler HAIRRELL, San Francisco, CA (US)

(73) Assignee: AURIS HEALTH, INC., Santa Clara, CA (US)

(21) Appl. No.: 19/233,591

(22) Filed: Jun. 10, 2025

Related U.S. Application Data
(60) Provisional application No. 63/684,821, filed on Aug. 19, 2024.

(51) Int. Cl. A61B 34/30; B25J 9/00

(52) U.S. Cl. CPC A61B 34/30; B25J 9/0012; A61B 2034/301

(57) ABSTRACT

A medical instrument includes an elongate shaft having a base at a proximal end of the elongate shaft. A robotic system can robotically articulate the elongate shaft by transferring drive outputs from a manipulator to drive inputs of the base. A working channel tool assembly can provide robotic and manual control of a medical tool insertable into a working channel formed as a lumen in the elongate shaft. The working channel tool assembly can couple to the base and the combination can be operated as a single unit, which may be attachable on an end effector. The working channel tool assembly can include a chassis, retainer(s), shuttle(s), latch, spool(s), any other components that are designed to provide structural support and organization for one another when assembled. The working channel tool assembly reduces use of fasteners and adhesives through various structural feature on each of the components.

### BACKGROUND

Certain robotic medical procedures can involve the use of shaft-type instruments, such as endoscopes, which may be inserted into a patient through an orifice (e.g., a natural orifice) and advanced to a target anatomical site. Such medical instruments can include a working channel and a medical tool may be introduced through the working channel to the target anatomical site.

### SUMMARY OF THE DISCLOSURE

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

In some aspects, the techniques described herein relate to a working channel tool assembly including: a chassis having thereon a first side rail and a first assembly interface; a retainer having thereon a second side rail and a second assembly interface, the second assembly interface configured to mate with the first assembly interface; and a first track formed by the first side rail and the second side rail when the chassis couples with the retainer via pressing the first assembly interface to the second assembly interface.

In some aspects, the techniques described herein relate to an assembly, wherein each of entirety of the chassis and entirety of the retainer is a single molded plastic.

In some aspects, the techniques described herein relate to an assembly, further including: a first shuttle having at least two opposing hooked edges, the first shuttle slidably coupled to the first track with the hooked edges.

In some aspects, the techniques described herein relate to an assembly, wherein the shuttle is fixedly coupled with a portion of a tool for insertion into a lumen of an endoscope, wherein sliding the first shuttle advances or retracts the tool within the lumen of the endoscope.

In some aspects, the techniques described herein relate to an assembly, wherein the tool is at least one of a laser tool or a basket tool.

In some aspects, the techniques described herein relate to an assembly, further including: a casing to internally house the chassis, the retainer, and the first shuttle, wherein the first shuttle has an attachment interface configured to mount an external component through a slot formed on the casing, the slot extending parallel the track.

In some aspects, the techniques described herein relate to an assembly, wherein the first shuttle has thereon a guide well to thread a string through.

In some aspects, the techniques described herein relate to an assembly, wherein the first shuttle has thereon a glue well that exposes a portion of a string, receives glue into the glue well and on the portion of the string, and adheres the string to the first shuttle.

In some aspects, the techniques described herein relate to an assembly, further including: a second track formed by a third side rail and a fourth side rail; and a second shuttle slidably coupled to the second track.

In some aspects, the techniques described herein relate to an assembly, wherein: the first shuttle is fixedly coupled with a first string controlling insertion and retraction of a first part of a tool, and the second shuttle is fixedly coupled with a second string controlling insertion and retraction of a second part of the tool.

In some aspects, the techniques described herein relate to an assembly, wherein: the first part of the tool is a basket sheath of a basket tool, and the second part of the tool is at least one wire configured to telescopically slide within and in relation to the basket sheath.

In some aspects, the techniques described herein relate to an assembly, wherein the chassis includes walls at ends of the first side rail, the walls configured to limit movement range of a component coupled to the first side rail.

In some aspects, the techniques described herein relate to an assembly, wherein the chassis includes a latch opening configured to pass through a locking mechanism of a latch.

In some aspects, the techniques described herein relate to an assembly, wherein the second assembly interface is a guide post configured to guide a string.

In some aspects, the techniques described herein relate to an assembly, wherein the retainer has molded there on an axle for rotation of a spool and a stopper to limit a range of the rotation.

In some aspects, the techniques described herein relate to an assembly, further including a spool, wherein the spool includes a capstan feature to fix an end of a string wound around the spool to the capstan feature.

In some aspects, the techniques described herein relate to a working channel tool assembly including: a first casing having thereon a first assembly interface; a second casing having thereon a second assembly interface; a latch including: a hinge; a biasing element; and a locking interface; and at least one component configured to control insertion or retraction of a tool within a working channel of an elongate shaft coupled to a base, wherein the first casing and the second casing limits a range of movement for the latch to rotation about the hinge when the first casing couples with the second casing via pressing the first assembly interface to the second assembly interface, and wherein the biasing element defaults the latch a locking configuration when the locking interface engages with a portion of the base.

In some aspects, the techniques described herein relate to an assembly, further including: a release interface, wherein when the release interface is interacted with, the biasing element deforms to rotate the latch to a release configuration such that the locking interface stops engaging the portion of the base.

In some aspects, the techniques described herein relate to an assembly, wherein the biasing element is a molded plastic having elastic property and the locking interface is a hook.

In some aspects, the techniques described herein relate to an assembly, wherein the biasing element is a deformable arc that changes curvature.

In some aspects, the techniques described herein relate to a shuttle including: a pair of hooked edges configured to hook onto opposing rails of a track, the hooked edges formed on a first side of a molded plastic; a string guide configured to provide string routing; an adhesive site to which an adhesive can be applied to fix the string to the shuttle; and an attachment interface configured to mount an external component, wherein the shuttle is housed within a first side of a casing, and wherein the attachment interface is configured to mount the external component on a second side of the casing opposing the first side.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example of a robotic medical system including a shaft-type instrument coupled to a robotic end effector, in accordance with some implementations.

FIGS. 2A and 2B illustrate medical system components that may be implemented in the medical system of FIG. 1, in accordance with some implementations.

FIG. 3 illustrates a shaft-type instrument disposed in a subject, in accordance with some implementations.

FIG. 4 shows a view of an example working channel tool assembly, in accordance with some implementations.

FIG. 5 illustrates an exploded view of the example working channel tool assembly shown in FIG. 4, in accordance with some implementations.

FIG. 6 illustrates a block diagram of example working channel tool assembly components, in accordance with some implementations.

FIGS. 7A-7C illustrate various views of an example chassis, in accordance with some implementations.

FIGS. 8A-8C illustrate various views of example retainers, in accordance with some implementations.

FIGS. 9A-9B illustrate various views of a first example shuttle, in accordance with some implementations.

FIGS. 10A-10C illustrate various views of a second example shuttle, in accordance with some implementations.

FIG. 11 illustrates a view of an example assembly of the chassis, retainers, and shuttles, in accordance with some implementations.

FIGS. 12A-12C illustrate various views of an example latch, in accordance with some implementations.

FIGS. 13A-13B illustrate the example latch of FIGS. 12A-12C in, respectively, a locked configuration and a released configuration, in accordance with some implementations.

FIGS. 14A-14C illustrate various views of an example spool, in accordance with some implementations.

FIG. 15 illustrates a flow diagram for an example process for assembling a working channel tool assembly, in accordance with some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts, features associated with separate figures that are identified by common reference numbers are not related and/or similar with respect to at least certain aspects.

The present disclosure provide systems, devices, and methods for assembling various parts that form a working channel tool assembly and for implementing various function/features within those parts. The working channel tool assembly can provide robotic and manual control of a medical tool insertable into a working channel (a lumen) of a shaft-like medical instrument. The shaft-like medical instrument can be navigated to a site within a subject and the working channel tool assembly can introduce a medical tool (e.g., a basket tool, a laser tool, forceps, imaging device, etc.) into the working channel such that the medical tool can advance to the site. The working channel tool assembly can include a chassis, retainer(s), shuttle(s), latch, spool(s), and any other components that, when assembled, are designed to provide structural support and organization for one another. Advantageously, the working channel tool assembly can reduce use of fasteners and adhesives through various structural features on each of the components. With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “shaft” is used herein according to its broad and ordinary meaning and may refer to any type of elongate cylinder, tube, scope (e.g., endoscope), prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope.

### Medical Procedures

Although certain aspects of the present disclosure are described in detail herein in the context of renal, urological, and/or nephrological procedures, such as kidney stone removal/treatment procedures, it should be understood that such context is provided for convenience and clarity, and working channel tool assembly concepts and designs disclosed herein are applicable to any suitable robotic medical instruments used in various medical procedures, such as robotic bronchoscopy. However, as mentioned, description of the renal/urinary anatomy and associated medical issues and procedures is presented below to aid in the description of the inventive concepts disclosed herein.

In certain medical procedures, such as ureteroscopy procedures, elongate medical instruments that access the treatment site through an access sheath may be utilized to remove debris, such as kidney stones and stone fragments or other refuse or contaminant(s), from the treatment site. Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis.” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shock wave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or aspiration.

In some procedures, surgeons may insert an endoscope (e.g., ureteroscope) into the urinary tract through the urethra to remove urinary stones from the bladder and ureter. Typically, a ureteroscope includes a camera proximate its distal end configured to enable visualization of the urinary tract. The ureteroscope can also include, or allow for placement in a working channel of the ureteroscope, a lithotripsy device configured to capture or break apart urinary stones. During a ureteroscopy procedure, one physician/technician may control the position of the ureteroscope, while another physician/technician may control the lithotripsy device(s).

In some procedures, such as procedures for removing relatively large stones/fragments, physicians may use a percutaneous nephrolithotomy (“PCNL”) technique that involves inserting a nephroscope through the skin (i.e., percutaneously) and intervening tissue to provide access to the treatment site for breaking-up and/or removing the stone(s). A percutaneous-access device (e.g., nephroscope, sheath, sheath assembly, and/or catheter) used to provide an access channel to the target anatomical site (and/or a direct-entry endoscope) may include one or more fluid channels for providing irrigation fluid flow to the target site and/or aspirating fluid from the target site (e.g., through passive outflow and/or active suction).

For ureteroscopic procedures, a physician may implement a procedure to break a relatively large kidney stone into a relatively smaller fragments to facilitate extraction thereof. For example, certain instruments may be utilized to break the stone into smaller fragments, such as by lasing, or through other application of cleaving force to the kidney stone. According to some procedures, a basketing device/system may be used to capture the relatively smaller stone fragment(s) and extract them from the treatment site out of the patient. Generally, when a stone is captured, the surgeon may wish to quickly extract the stone through the ureteral access sheath prior to opening the basket to deposit/drop the stone into a specimen collection structure or area, after which the basket may be closed and reinserted (e.g., within a working channel of an endoscope/ureteroscope) through the access sheath for the purpose of extracting remaining stones or stone fragments, should there be any.

Robotic-assisted ureteroscopic procedures can be implemented in connection with various medical procedures, such as kidney stone removal procedures, wherein robotic tools can enable a physician/urologist to perform endoscopic target access as well as percutaneous access/treatment. Advantageously, aspects of the present disclosure relate to systems, devices, and methods for robotically controlling insertion, retraction, and operation of various medical tools that can be navigated through a working channel of an endoscope.

### Medical System

Various aspects of the present disclosure described herein may be integrated into a robotically enabled/assisted medical system, including a surgical robotic system (robotic system for short), capable of performing a variety of medical procedures. Among endoscopy procedures, the robotically enabled medical system may be capable of performing bronchoscopy, ureteroscopy, gastroscopy, etc.

In addition to performing the breadth of procedures, the robotically enabled medical system may provide additional benefits, such as enhanced imaging and guidance to assist the medical professional. Additionally, the robotically enabled medical system may provide the medical professional with the ability to perform the procedure from an ergonomic position without the need for awkward arm motions and positions. Still further, the robotically enabled medical system may provide the medical professional with the ability to perform the procedure with improved ease of use such that one or more of the instruments of the robotically enabled medical system may be controlled by a single operator.

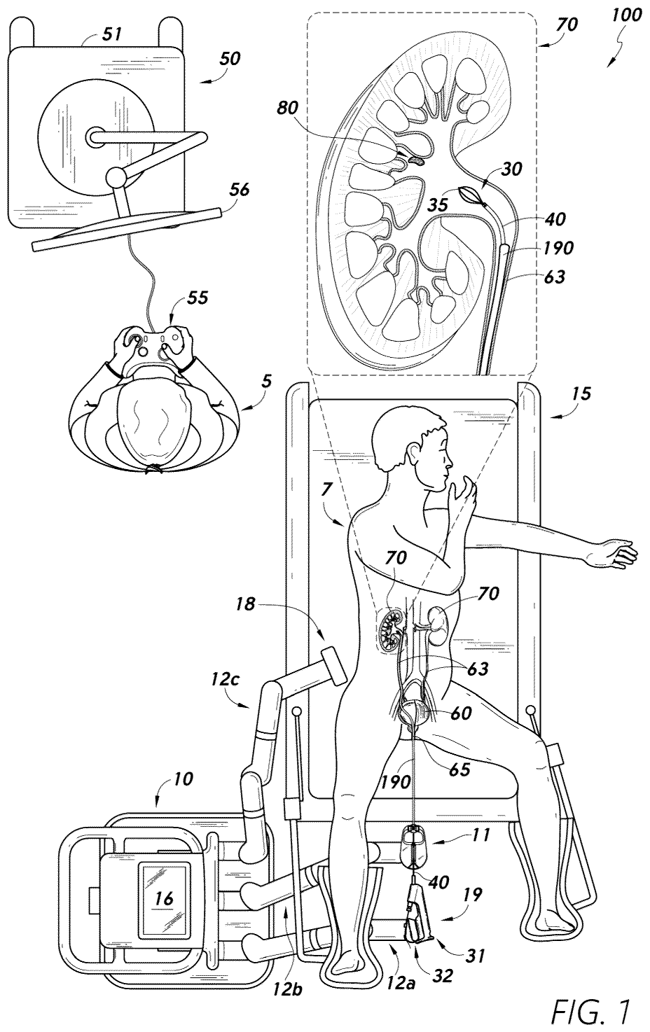

FIG. 1 illustrates an example medical system 100 for performing various medical procedures in accordance with aspects of the present disclosure. The medical system 100 may be used for, for example, endoscopic (e.g., ureteroscopic) procedures. The principles disclosed herein may be implemented in any type of endoscopic (e.g., bronchial, gastrointestinal, etc.) and/or percutaneous procedure.

The medical system 100 includes a robotic system 10 (e.g., a mobile robotic cart as shown, a table-based system with integrated robotic arms, etc.) that is configured to engage with and/or control a medical instrument 19 (e.g., ureteroscope) including a proximal instrument base 31 and a shaft 40 coupled to the instrument base 31 at a proximal portion thereof to perform a direct-entry procedure on a subject 7. The term “subject” is used herein to refer to live patient and human anatomy as well as any subjects to which the present disclosure may be applicable. For example, the “subject” may refer to physical anatomic models (e.g., anatomical education model, anatomical model, medical education anatomy model, etc.) used in dry runs, models in computer simulations, or the like that covers non-live patients or test subjects. The term “direct-entry” is used herein according to its broad and ordinary meaning and may refer to any entry of instrumentation through a natural or artificial opening in a patient's body. For example, with reference to FIG. 1, the direct entry of the scope/shaft 40 into the urinary tract of the subject 7 may be made via the urethra 65.

It should be understood that the direct-entry instrument 19 may be any type of shaft-based medical instrument, including an endoscope (such as a ureteroscope), catheter (such as a steerable or non-steerable catheter), nephroscope, laparoscope, or other type of medical instrument. The medical instruments disclosed herein may be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body.

The medical instrument 19 has a flexible elongated body that has mechanical couplings which enable the elongated body to flex, bend, deflect or articulate to some angle, in response to an actuator (e.g., containing a motor) being energized in accordance with a command (also referred to as an input, which refers to (e.g., defines) a desired direction or a desired articulation angle, for example). An example of such a medical instrument is a flexible endoscope (or scope) that may be any type of elongated medical instrument having image generating, viewing, and/or capturing functionality and configured to be introduced into any type of organ, cavity, lumen, chamber, or space of a patient's body. A scope may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. The elongated body may comprise a flexible tube or shaft and may be dimensioned to be passed within an outer sheath, catheter, introducer, or other lumen-type device, or it may be used without such devices.

In an example use case, if the subject 7 has a kidney stone (or stone fragment) 80 located in a kidney 70, the operator 5 may perform a procedure to remove the stone 80 through a urinary tract 63 using a basket 35. In some examples, the operator 5 can interact with the control system 50 and/or the robotic system 10 to cause/control the robotic system 10 to advance and navigate the medical instrument 19 through calyx network of the kidney 70 to where the stone 80 is located. The control system 50 can provide information via the display(s) 56 that is associated with the medical instrument 19, such as real-time endoscopic images captured therewith, and/or other instruments of the system 100, to assist the operator 5 in navigating/controlling such instrumentation.

The medical system 100 shown as an example in the figures further includes a table 15, and an electromagnetic (EM) field generator 18. Table 15 is configured to hold the subject 7 for example as shown. EM field generator 18 may be held by one of the robotic arms (robotic arm 12c) of the robotic system 10, may be a stand-alone device, or may be integrated into the table 15. In some versions, the table has actuators which can change, for example the height and orientation of the table 15. The control system 50 may communicate with the table 15 to position the table 15 in a particular orientation or otherwise control the table 15.

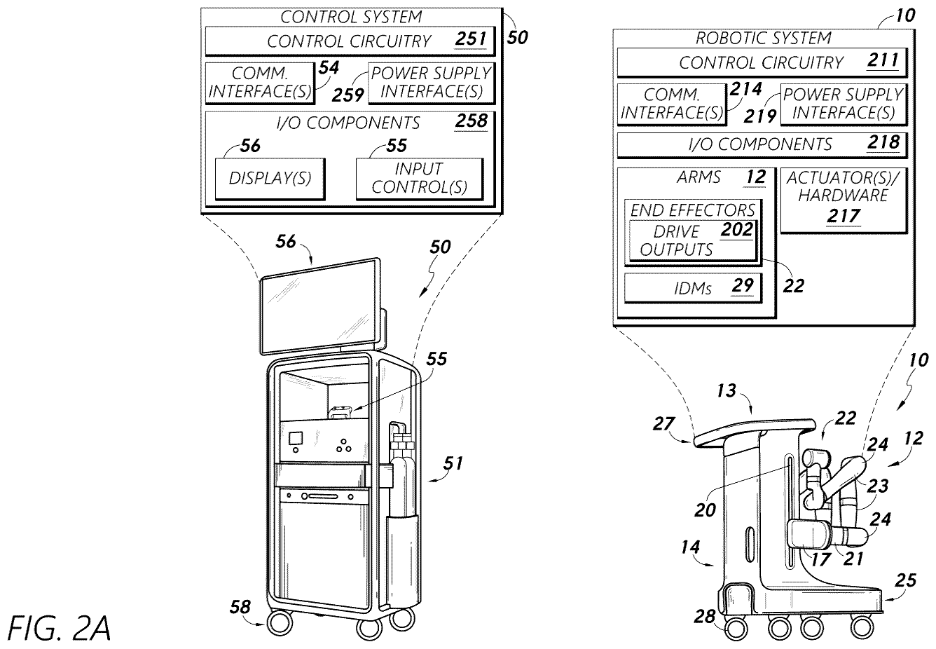

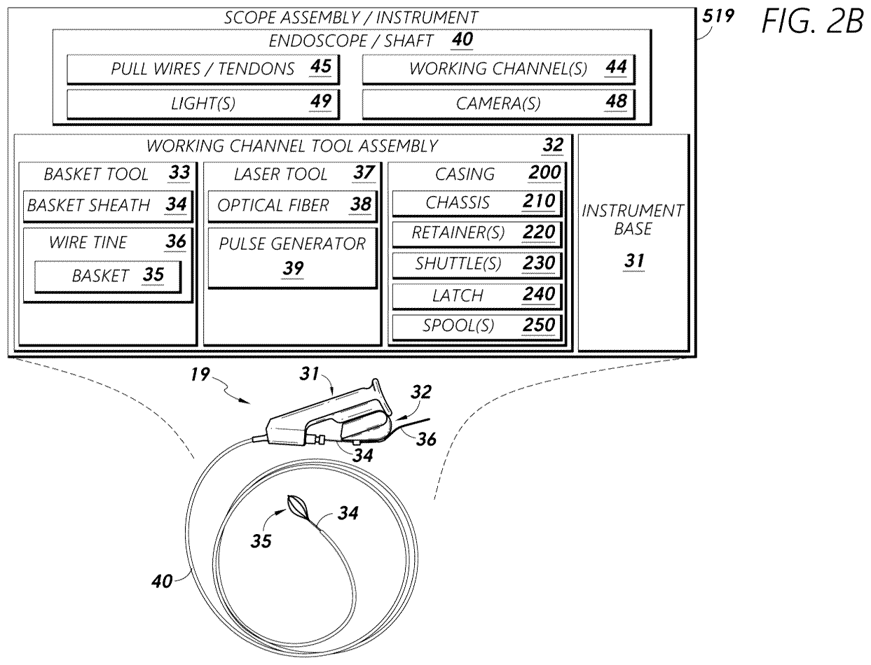

As shown in FIG. 2A, the medical system 100 of FIG. 1 can include a control system 50 configured to interface with the robotic system 10, provide information regarding the procedure, and/or perform a variety of other operations. The control system 50 of the present example includes various input/output (I/O) components 258 configured to assist an operator or others in performing a medical procedure. For example, with reference to FIG. 1, the I/O components 258 may be configured to allow for user input from operator 5 to control/navigate the medical instrument 19 and any components thereof within the subject 7. I/O components 258 of the present example include one or more input controllers 55 configured to receive user input and one or more displays 56 configured to present certain information to the user. Controller 55 may take any suitable form, including but not limited to one or more buttons, keys, joysticks, handheld controllers (e.g., video-game-type controllers), computer mice, trackpads, trackballs, control pads, foot pedals, and/or sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 50 of the present example includes a communication interface 54 that is configured to provide a communicative interface between control system 50 and robotic system 10, medical instrument 19, and/or other components. Communications via communication interface 54 may include data, commands, electrical power, and/or other forms of communication. Communication interface 54 may also be configured to provide communication via wire, wirelessly, and/or other modalities. Control system 50 also includes a power supply interface 259, which may receive power to operate control system 50 via wire, battery, and/or any other suitable kind of power source. A control circuitry 251 of the control system 50 may provide signal processing and execute control algorithms to achieve the functionality of the medical system 100 as described herein.

The control system 50 may also communicate with the robotic system 10 to receive data therefrom relating to the robotic system and/or the medical instrument attached thereto, for example, the pose (e.g., position and/or orientation) of the distal end of the medical instrument flexible elongated body. Such pose data may be derived using one or mor position sensors (e.g., EM sensors) that may be mounted to, integrated with, or otherwise attached to the flexible elongated body of the medical instrument 19, and that interact with an EM field generated by the EM field generator 18. The control system 50 may communicate with the EM field generator 18 to control the generation of the EM field in an area around the subject 7. Other ways of detecting the pose (e.g., 3D position and/or orientation) of the distal end of the medical instrument 19 are possible, such as using an optical camera/imaging-based system.

As noted above and as shown in FIG. 1 and FIG. 2A, the robotic system 10 includes robotic arms 12 that are configured to engage with and/or control the medical instrument 19 to perform one or more aspects of a procedure. It should be understood that a robotic arm 12 may be coupled to instruments that are different than those shown in FIG. 1 (e.g., introducer for guiding the medical instrument 19 to an access point, such as the urethra 65); and in some scenarios, one or more of the robotic arms 12 may not be utilized or coupled to a medical instrument. Each robotic arm 12 includes multiple arm segments 23 coupled to joints 24, which enable the attached medical instrument to have multiple degrees of movement/freedom. In the example of FIG. 1, the robotic system 10 is positioned proximate to the patient's legs and the robotic arms 12 are actuated to engage with and position the medical instrument 19 for access into an access opening of the subject 7. When the robotic system 10 is properly positioned, the medical instrument 19 may be inserted into the subject 7 robotically using the robotic arms 12, manually by the operator 5, or a combination thereof.

The robotic system 10 may be coupled to any component of the medical system 100, such as the control system 50, the table 15, the EM field generator 18, the medical instrument 19, and/or any type of percutaneous-access instrument (e.g., needle, catheter, nephroscope, etc.). As noted above, the robotic system 10 may be communicatively coupled with the control system 50 via communication interfaces 214, 54. The robotic system 10 also includes a power supply interface 219, which may receive power to drive robotic system 10 via wire, battery, and/or any other suitable kind of power source. In addition, the robotic system 10, in one example, includes various input/output (I/O) components 218 configured to assist the operator 5 or others in performing a medical procedure. Such I/O components 218 may include any of the various kinds of I/O components 258 described later with greater detail in the context of the control system 50. In addition, or in the alternative, I/O components 218 of robotic system 10 may take any suitable form (or may be omitted altogether).

The robotic system 10 of the present example generally includes a robotic system base 25, a column 14, and a console 13 at the top of the column 14. The robotic system base 25 balances the weight of the column 14, arm support 17, and arms 12 over the floor. Accordingly, the robotic system base 25 may house certain relatively heavier components, such as electronics, motors, power supply, as well as components that selectively enable movement or immobilize the robotic system. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 10 in place during the procedure.

Positioned at the upper end of column 14, the console 13 can provide both a user interface (e.g., I/O components 218) for receiving user input and a display screen 16 (or a dual-purpose device such as, for example, a touchscreen) to provide the physician/user with both pre-operative and intra-operative data. As shown, the console 13 can also include a handle 27 to assist with maneuvering and stabilizing the robotic system 10. The column 14 may include one or more arm supports 17 for supporting the deployment of the one or more robotic arms 12 (where three robotic arms 12a, 12b, 12c are shown in FIG. 1). The arm support 17 may include individually-configurable arm mounts that rotate along one or more axes, including a perpendicular axis, to adjust the base of the robotic arms 12 for desired positioning relative to the subject 7.

The arm support 17 may be configured to vertically translate along the column 14. In some examples, the arm support 17 can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, subject sizes, and physician preferences. Similarly, the individually-configurable arm mounts on the arm support 17 can allow the robotic arm base 21 of robotic arms 12 to be angled in a variety of configurations.

The robotic arms 12 may generally comprise robotic arm bases 21 and end effectors 22, separated by a series of linking arm segments 23 that are connected by a series of joints 24, each joint comprising one or more independent actuators 217. Each actuator may comprise an independently-controllable motor. Each independently-controllable joint 24 can provide or represent an independent degree of freedom available to the robotic arm. In some examples, each of the arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the subject 7 to create greater access, while avoiding arm collisions.

The term “end effector” is used herein according to its broad and ordinary meaning and may refer to any type of robotic manipulator device, component, and/or assembly. Where an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly, wherein such assembly may or may not also include a medical instrument (or instrument handle/base) physically coupled to the adapter and/or end effector. The terms “robotic manipulator” and “robotic manipulator assembly are used according to their broad and ordinary meanings, and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, sterile adapter, and/or other component(s).

The end effector 22 of each of the robotic arms 12 may include one or more instrument device manipulator (IDMs 29), which may be attached using a mechanism changer interface (MCI). The MCI may provide power and control interfaces (e.g., connectors to transfer pneumatic pressure, electrical power, electrical signals, and/or optical signals from the robotic arm 12) to the IDM 29. In some examples, the IDM 29 may be removed and replaced with a different type of IDM 29, depending on the type of the medical instrument 19 that is to be attached to the arm. Each type of IDM 29 may serve to manipulate a respective type or a portion of the medical instrument 19. In the case where the medical instrument is a scope, the IDM 29 may use any one or combination of techniques including, for example, direct drives, harmonic drives, geared drives, belts and pulleys, magnetic drives, and the like, to drive the flexible elongated body of the scope so that the distal end is positioned to some desired angle or bent in some desired direction. A second type of IDM 29 may manipulate a basketing system or a steerable catheter by driving the flexible elongated body of the catheter or basketing system so that the distal end is positioned at some angle. Another type of IDM 29 may be configured to hold the EM field generator 18. Many variations are possible.

The medical system 100 of FIG. 1 may include certain control circuitry configured to perform certain of the functionality described herein, including the control circuitry 211 of the robotic system 10 and the control circuitry 251 of the control system 50. That is, the control circuitry of the medical system 100 may be part of the robotic system 10, the control system 50, or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational/executable instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry 211, 251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

In some examples, for example, the operator 5 may provide input to the control system 50 and/or robotic system 10; and in response to such input, control signals may be sent to the robotic system 10 to manipulate the medical instrument 19. The control system 50 may include one or more display devices (e.g., the display 56) to provide various information regarding a procedure. For example, the display 56 may provide information regarding the medical instrument 19. In the case of a scope, the control system 50 may receive real-time images that are captured by the scope representing internal anatomy of the subject 7 and display the real-time images via the display 56.

FIG. 2B illustrates a scope assembly 519 as the medical instrument 19 shown in FIG. 1, in accordance with some implementations. The endoscope (i.e., “scope” or “shaft”) can include an elongate shaft including one or more lights 49 and one or more cameras or other imaging devices 48, which may be integrated as a part of the endoscope or provided as a separate camera assembly. The scope 40 can further include one or more working channels 44, which may run a length of the scope 40. In some examples, such channel(s) may be utilized to provide access for a working channel tool (e.g., a basket tool 33, a laser tool 37, forceps, the camera assembly, etc.) for introduction through the scope 40 to the treatment site.

The scope assembly 519 may comprise an instrument base 31 for the scope 40, wherein the scope 40 is coupled to the instrument base 31 at a proximal end of the scope 40. The instrument base 31 can be shaped and configured as a handle to provide scope control either manually or robotically. In some examples, a working channel tool assembly 32 may releasably attach to the instrument base 31 and be configured to provide control of the working channel tool, either manually or robotically, in relation to the working channel 44 of the scope 40. When attached, the instrument base 31 and the working channel tool assembly 32 can operate as a single instrument unit (referred herein as an instrument handle) to be mounted on and operated by an end effector 22 of a single robotic arm 12a shown in FIG. 1 and FIG. 2A. The releasable attachment supports modular design of the instrument handle that, advantageously, help isolate working channel tool control from the scope control.

In some examples, the working channel tool assembly 32 may be configured to drive/control various interchangeable tools. An example tool is a basket tool 33 (e.g., a basketing assembly) which may comprise a basket 35 formed of one or more wire tines 36. For example, the basket tool 33 may comprise four wire tines 36 disposed within a basket sheath 34 over a length thereof, wherein the tines 36 project from a distal end of the basket sheath 34 to form the basket 35. The wire tines 36 further extend from the proximal end of the basket sheath 34, as shown. The wire tines 36 and the basket sheath 34 can be coupled to respective drive inputs (not shown) of the working channel tool assembly 32. The wire tines 36 may be configured to be slidable within the basket sheath 34, subject to some amount of frictional resistance, and be biased toward expansion of the basket 35 at a distal end of the basket sheath 34. When the wire tines 36 are extended out of the basket sheath 34 at the distal end (more of the wire tines 36 are pushed into the basket sheath 34), the basket 35 can open. Conversely, when the wire tines 36 are retracted into the basket sheath 34 at the distal end (more of the wire tines 36 are pulled out of the basket sheath 34), the basket 35 can close.

Another example tool is a laser tool 37 which may be introduced into the working channel 44, driven to the tissue site, and perform lithotripsy. The laser tool may comprise an optical fiber 38 to guide the laser and a pulse generator 39 for energy transfer that would fragment a kidney stone in ureteroscopy. In some examples, after fragmentation, the laser tool 37 may be pulled out of the working channel 44 and the basket tool 33 may be introduced into the working channel 44 to remove the kidney stone fragments.

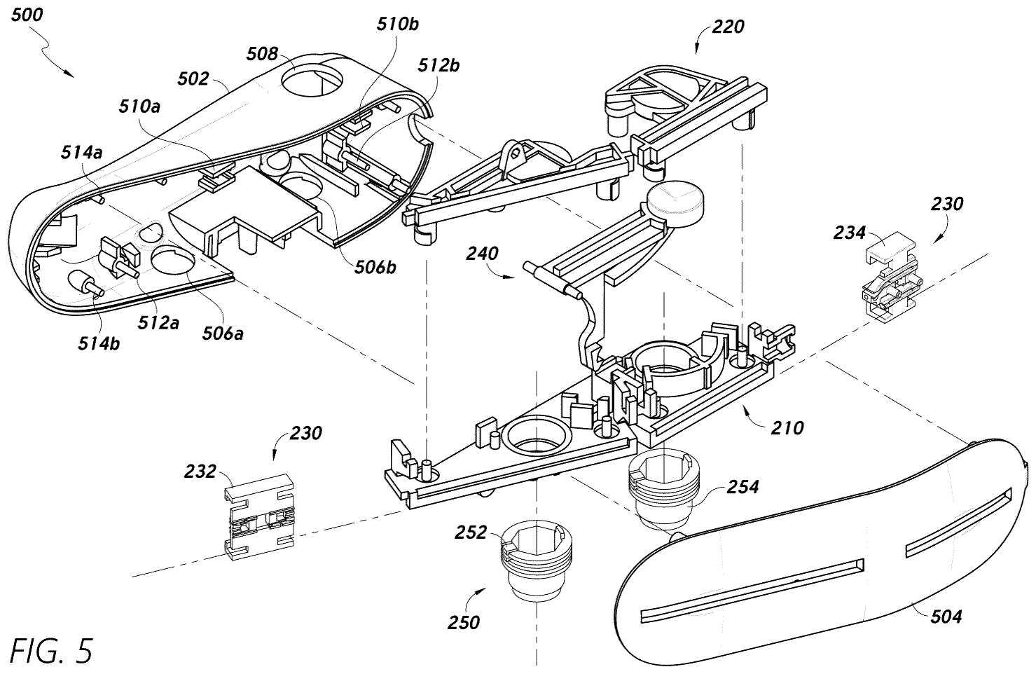

Structurally, the working channel tool assembly 32 can include a casing (housing) 200 that contains various components including: a chassis 210, at least one retainer(s) 220, at least one shuttle(s) 230, a latch 240, and at least one spool(s) 250. The components and their relationships to another component are shown and described in relation to FIG. 5. Any of the components may be molded as a single continuous plastic.

The scope assembly 519 can be powered through a power interface 79 and/or controlled through a control interface 78, each or both of which may interface with a robotic arm/component of the robotic system 10. For example, with reference to FIG. 1, an instrument feeder 11 can be attached to the distal end effector 22 of one of the arms 12b to facilitate robotic control/advancement of the scope 40. The scope assembly 519 can be attached to the distal end effector 22 of another one of the arms 12a to facilitate control/advancement of the instrument base 31 and the working channel tool assembly 32.

The various components of the medical system 100 of FIG. 1 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces 54 and 214 of the systems of FIG. 2A can be configured to communicate with one or more device/sensors/systems, such as over a wireless and/or wired network connection. In some examples, the various communication interfaces can implement a wireless technology such as Bluetooth, Wi-Fi, near-field communication (NFC), or the like. Furthermore, in some examples, the various components of the system 100 can be connected for data communication, fluid exchange, power exchange, and so on via one or more support cables, tubes, or the like.

### Attachment and Operation of Working Channel Tool Assembly

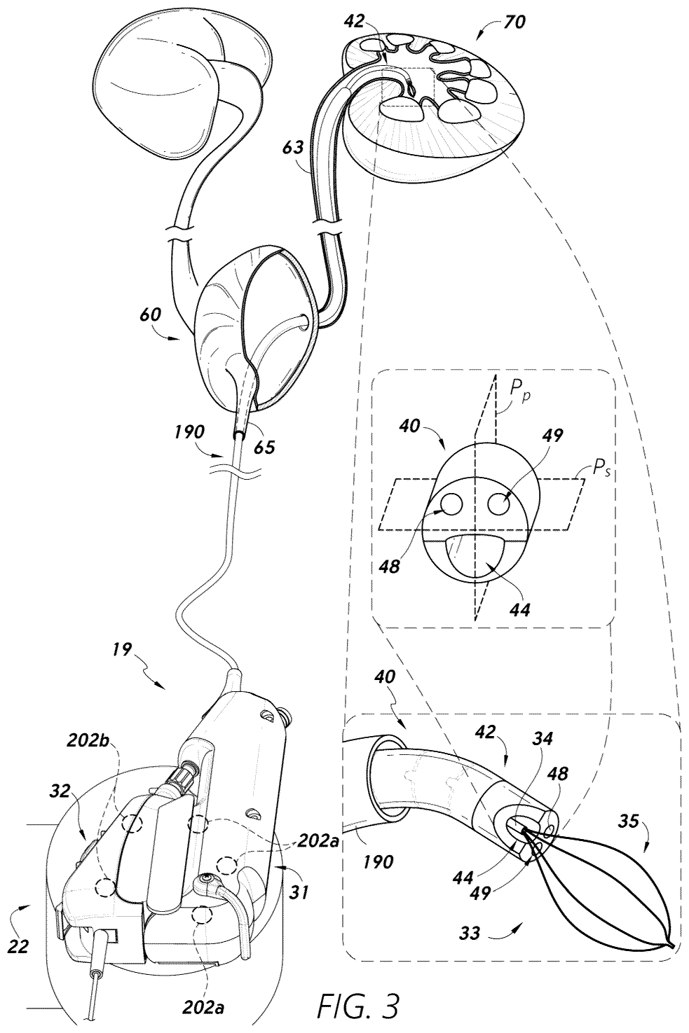

FIG. 3 illustrates an example ureteroscope (e.g., the scope assembly 519 of FIG. 2B) disposed in portions of the urinary system of a subject, in accordance with some implementations. As referenced above, ureteroscopic procedures can be implemented for investigating abnormalities in human urinary system, such as ureters and kidneys, and/or treating the same. For example, ureteroscope procedures can be implemented to treat and/or remove kidney stones. Such procedures may be implemented manually at least in part and/or may be performed using robotic technologies at least in part. For example, use of robotic devices and/or systems for certain endoscopic procedures can provide relatively greater precision, control, and/or coordination compared to strictly manual procedures.

The scope assembly 519 may comprise a tubular and flexible medical shaft/instrument as a scope 40 configured to be inserted into the anatomy of a subject to capture images of the anatomy and to perform certain tasks using one or more working channels 44 to deploy a variety of working channel tools (e.g., FIG. 2B's the basket tool 33, the laser tool 37, etc.). In some examples, the scope 40 can accommodate wires and/or optical fibers to transfer signals to/from an optical assembly and a distal end 42 of the scope 40, which can include an imaging device 48, such as an optical camera. The scope 40 can further include a light source 49, such as an LED or fiber-optic light source/lens.

The scope 40 can be advanced to the target location through an access sheath 190. The access sheath 190 may be advanced through the urethra 65, bladder 60, and urinary tract 63 to a kidney 70. The distal end of the access sheath 190 may be parked at a position in the urinary tract 63. The access sheath 190 may be placed as far into the renal anatomy as possible, as permitted by the urinary tract 63 path, which may be somewhat tortuous in certain portions thereof.

The scope 40 can be articulable, such as with respect to at least a distal portion 42 of the scope 40, so that the scope 40 can be steered within the human anatomy. In some examples, the scope 40 is configured to be articulated with, for example, six degrees of freedom, including XYZ coordinate movement, as well as pitch, yaw, and roll. Certain position sensor(s) (e.g., electromagnetic sensors) of the scope 40, where implemented, may likewise have similar degrees of freedom with respect to the positional information they generate/provide.

For robotic implementations, robotic arms (e.g., the robotic arm(s) 12) of a robotic system can be configured/configurable to manipulate the scope 40. For example, an end effector of a robot arm can be coupled to proximal ends of elongate movement members extending along the length of the scope 40 and manipulate the scope 40 using the elongate movement members. The elongate movement members may include one or more pull wires (e.g., pull or push wires), cables, fibers, and/or flexible shafts. For example, the robotic end effector may be configured to actuate multiple pull wires (not shown) coupled to the scope 40 to deflect the tip 42 of the scope 40. Pull wires may include any suitable or desirable materials, such as metallic and non-metallic materials such as stainless steel, Kevlar, tungsten, carbon fiber, and the like. In some examples, the scope 40 is configured to exhibit nonlinear behavior in response to forces applied by the elongate movement members. The nonlinear behavior may be based on stiffness and compressibility of the scope, as well as variability in slack or stiffness between different elongate movement members.

The scope 40 can be deflectable in one or two directions within a first/primary plane Pₚ. The scope 40 can also be deflectable in one or two directions in a second/secondary plane Pₛ, which may be orthogonal to the primary plane Pₚ. For example, it can be desirable for the at least the distal section 42 of the scope 40 to be deflectable in more than one plane to reach the desired area, such as the specific anteriorly or posteriorly pointing calyx. Although the primary Pₚ and secondary Pₛ deflection planes are shown in a particular configuration, it should be understood that the illustrated secondary plane Pₛ may be the primary plane Pₚ and vice versa.

The instrument base 31 can generally include an attachment interface having one or more mechanical inputs (e.g., receptacles, pulleys, spools, female inputs, etc.) that are designed to be reciprocally mated with one or more torque couplers on an attachment surface of an end effector 22. In some examples, the instrument base 31 can be configured to attach, mount, or otherwise be connected or coupled to the robotic end effector 22 and one or more drive outputs thereof. The instrument base 31 can include one or more drive inputs configured to engage with and be actuated by corresponding drive outputs to manipulate the scope 40. The elongate movement members can be coupled to the drive inputs and the drive inputs can be configured to control or apply tension to the elongate movement members in response to drive outputs.

Previously in relation to FIG. 2A, it was described that a working channel tool assembly 32 can couple to the instrument base 31 and be operate as a single instrument handle. Further, it was described that the instrument handle can be mounted on and operated by an end effector 22 of a single robotic arm (e.g., the robotic arm 12a in FIG. 1). Like the instrument base 31, the working channel tool assembly 32 can be configured to attach, mount, or otherwise be connected or coupled to the robotic end effector 22 and its drive outputs thereof. Like the instrument base 31, the working channel tool assembly 32 can include one or more drive input(s) to manipulate a working channel tool.

The coupling and the mounting of the instrument base 31 and the working channel tool assembly 32 as a single instrument handle can advantageously help align all of the instrument unit and maximally utilize drive outputs 202 of the end effector 22. For example, the example end effector 22 shown has five drive outputs where a first set of drive outputs 202a are engaged with drive inputs of the instrument base 31 and a second set of drive outputs 202b are engaged with drive inputs of the working channel tool assembly 32. Each of the first set of drive outputs can be configured to control one of first deflections on Pₚ, second deflections on Pₛ, and shaft roll. In FIG. 3, the working channel tool assembly 32 is configured with a basket tool (e.g., the basket tool 33 of FIG. 2B). Each of the second set of drive outputs 202b can be configured to control one of insertion/retraction and open/close of the basket tool.

The present disclosure focuses on various aspects of the working channel tool assembly 32 including anatomy, operation, and features/functionalities thereof. Following figures and their related description provide additional details.

### Components of Working Channel Tool Assembly

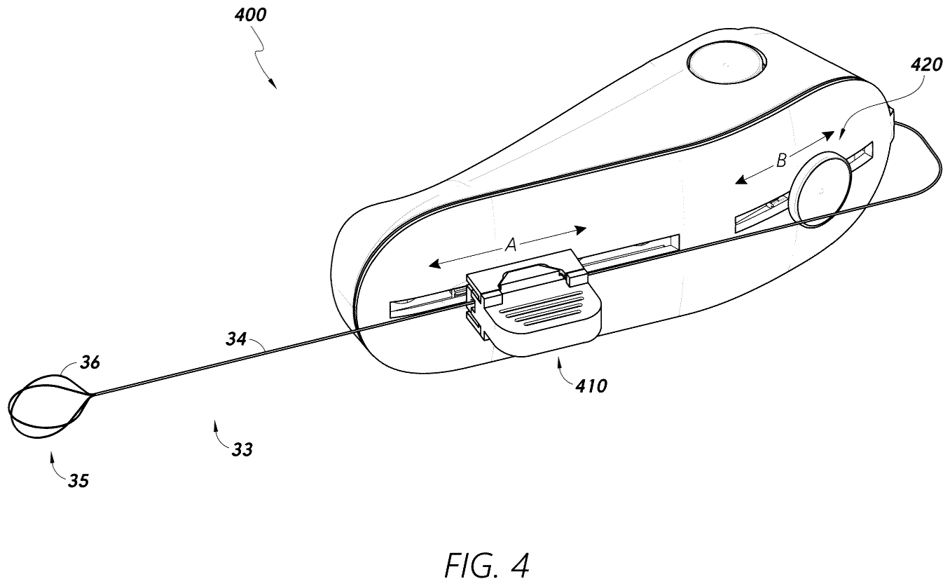

FIG. 4 shows a view 400 of an example working channel tool assembly (e.g., the working channel tool assembly 32 of FIG. 2B), in accordance with some implementations. The working channel tool assembly may include one or more interfaces to facilitate manual control of working channel tool functions. For example, the working channel tool assembly shown has a first interface 410 slidable along a first axis ‘A’ and a second interface 420 slidable along a second axis ‘B’, although other interfaces with other mechanisms are possible.

Manual sliding of the first interface 410 “forward” (toward the basket 35 as shown) can advance a working channel tool fixed to the first interface 410 toward a target site. Conversely, sliding “backward” (away from the basket 35 as shown) can retract the fixed working channel tool away from the target site. Similarly, manual sliding of the second interface 420 can advance or retract another working channel tool fixed to the second interface 420 toward or away from the target site. For example, a laser driver fixedly attached to the first interface 410 may advance into a target site when the first interface 410 slides forward, and retracts from the target site when the first interface 410 slides backward.

In some examples, the interfaces 410, 420 can be coupled to different parts of the same working channel tool to effectuate relative motion of the parts to each other. FIG. 4 shows a basket tool 33 comprising wire tines 36 telescopically slidable within a basket sheath 34 and forming a basket 35. Here, the first interface 410 can be fixedly coupled with the basket sheath 34 and the second interface 420 can be fixedly coupled with the proximal ends of the wire tines 36. The wire tines 36 are configured to be slidable within the basket sheath 34, subject to some amount of frictional resistance. When the first interface 410 fixed to the access sheath 34 slides, due to the frictional resistance, it may advance both the basket sheath 34 and the wire tines 36. In contrast, when the second interface 420 fixed to the wire tines 36 slides, the wire tines 36 may overcome the frictional resistance and relatively advance or retract in relation to the stationary basket sheath 34.

As previously described, the wire tines 36 may be biased toward expansion of the basket 35 at a distal end of the basket sheath 34. When the wire tines 36 are pushed out of the basket sheath 34 with forward sliding of the second interface 420, the wire tines 36 can expand and cause the basket 35 to open. Conversely, when the wire tines 36 are retracted into the basket sheath 34 with backward sliding of the second interface 420, the wire tines 36 can contract and cause the basket 35 to close.

FIG. 5 illustrates an exploded view 500 of the example working channel tool assembly 32 shown in FIG. 4, in accordance with some implementations. The working channel tool assembly as shown is exemplary and may have additional or omitted components. For example, the first interface 410, the second interface 420, and the basket tool 33 shown in FIG. 4 are omitted in the interest of brevity.

The working channel tool assembly can include a first casing 502 and a second casing 504 opposing the first casing 502. The casings 502, 504 can be coupled to each other with use of fasteners, connectors, or other assembly interfaces. Some example assembly interfaces can include, without limitations, press pins (also known as dowel pins or drive pins), snaps, bayonet, pin-in-slot, or the like. The example tool assembly shown couples the first casing 502 with the second casing 504 using press pins 514a, 514b on the first casing 502 and corresponding receptors (not shown) on the second casing 504. Advantageously, such press pin coupling can eliminate need of additional fastening components, such as screws or rivets, thereby reduce material costs, and provide easier assembly.

The casings 502, 504 can provide structural support and guides for components to be contained therein. As examples of structural support, the casings 502, 504 can provide slots 510a, 510b to firmly hold the chassis 210 in place when the casings 502, 504 are coupled to each other. As examples of structural guides, the casing 502 includes a cutout 508 for a latch 240 and cylindrical features 506a, 506b configured to accommodate spools 250 (including a first spool 252 and a second spool 254).

The exploded view 500 additionally illustrates retainer(s) 220 and shuttles 230 (including a first shuttle 232 and a second shuttle 234). Each component shown in the exploded view 500 will be described in greater detail below.

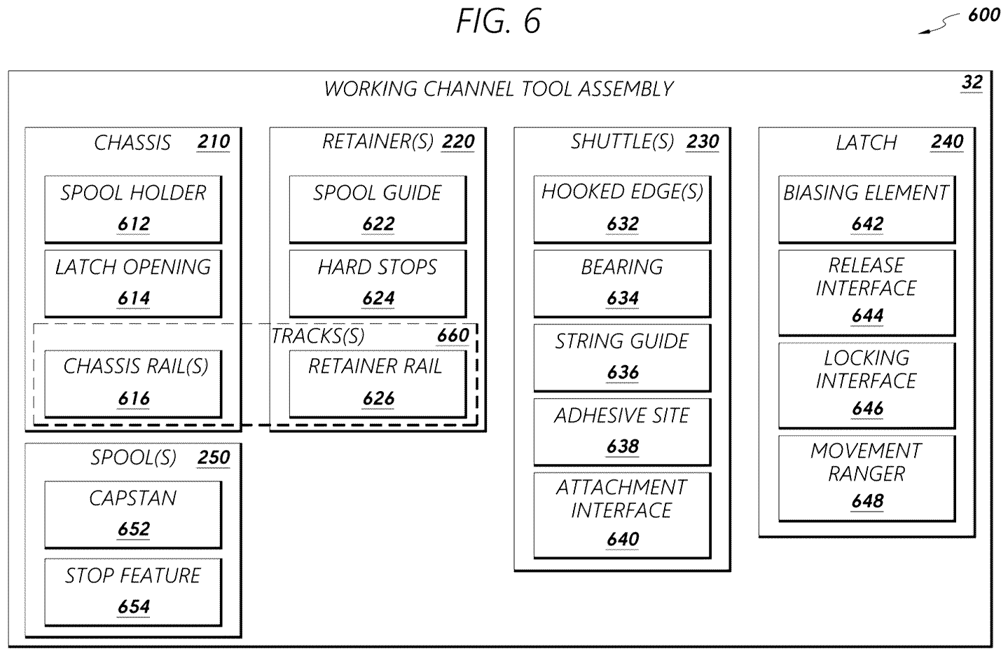

FIG. 6 illustrates a block diagram 600 of the example working channel tool components shown in FIG. 5, in accordance with some implementations. Some example components of the working channel tool assembly 32 may include a chassis 210, retainer(s) 220, shuttle(s) 230, a latch 240, and spool(s) 250.

The chassis 210 can be configured as a platform for attachment, stabilization, and organization of other components. The chassis 210 can include a spool holder 612, a latch opening 614, and chassis rail(s) 616. The spool holder 612 can be configured to accommodate the spool(s) 250. The latch opening 614 can be configured to allow at least a portion of the latch 240 through the chassis 210 and, in some examples, limit range of motion of the latch 240. The chassis rail(s) 616 can form one side of track(s) 660.

The retainer(s) 220 can be configured to couple with the chassis 210 and to provide various functions including guiding rotation of the spool(s) 250, guiding string(s) around their path as the rotation of the spool(s) 250 gets converted to translation, providing the other drive rail for the track(s) 660, or the like. In relation to these functionalities, the retainer(s) 220 can include a spool guide 622, hard stops 624, and a retainer rail 626. The spool guide 622 can provide guiding mechanisms (e.g., belts, pulleys, guide posts, etc.) for strings (e.g., pull wires). The retainer rail 626 can form the other side of the track(s) 660 by opposing the chassis rail(s) 616. The hard stops 624 can block or otherwise limit movement of the shuttle(s) 230 slidably coupled on the track(s) 660.

The shuttle(s) 230 can be configured to provide string routing, string termination on the shuttle(s) 230, external component mounting, bearing features for smooth translation along the track(s) 660. In relation to these functionalities, the shuttle(s) 230 can include hooked edge(s) 632, a bearing 634, a string guide 636, an adhesive site 638, and an attachment interface 640. The hooked edge(s) 632 can loosely lock the shuttle(s) 230 onto the track(s) 660 such that the shuttle(s) 230 may slide on the track(s) 660 while preventing the shuttle(s) 230 from falling off the track(s) 660. The bearing 634 can be a structural feature configured to provide smooth and controlled sliding of the shuttle(s) 230 on the track(s) 660 through reduction in friction, sticking, jerking, or the like during the sliding. The string guide 636 can align strings, which may be adhered to the shuttle(s) 230 at the adhesive site 638, with a sliding axis of the shuttle(s) 230 such that sliding of the shuttle(s) 230 on the track(s) 660 pulls the strings without causing entanglement. The attachment interface 640 can be configured to allow attachment of an interface (e.g., the interfaces 410, 420 of FIG. 4) to the shuttle(s) 230.

The latch 240 can be configured to provide a locking mechanism that releasably couples, with respect to FIG. 3, the working channel tool assembly 32 to the end effector 22. Referring back to FIG. 6, the latch 240 can include a biasing element 642, a release interface 644, a locking interface 646, and a movement ranger 648. The biasing element 642 can bias the locking interface 646 of the latch 240 toward providing/maintaining coupling to the end effector 22. The release interface 644 can be a part of the locking mechanism that allows release of the coupling via unbiasing of the locking interface 646. The movement ranger 648 can limit movement of the locking interface 646 caused by the biasing element 642 and operation of the release interface 644 such that the locking mechanism remains reusable.

The spool(s) 250 can be configured to provide a smooth bearing surface for rotational torque input, a fixation point for a string, a wrapping and securing structure for a string. The spool(s) 250 can include a capstan 652 and a stop feature 654. The capstan 652 can serve as a fixation point (termination point) for a string. The stop feature 654 can limit the rotation of the spool(s) 250 to within a range.

Each component is described in greater detail below in relation to various views of each component.

### Chassis

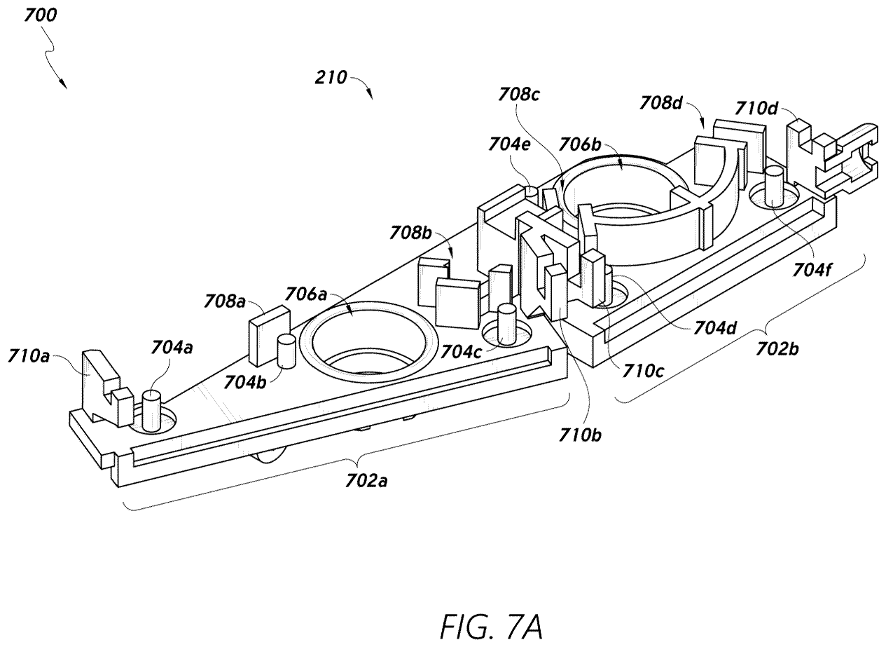

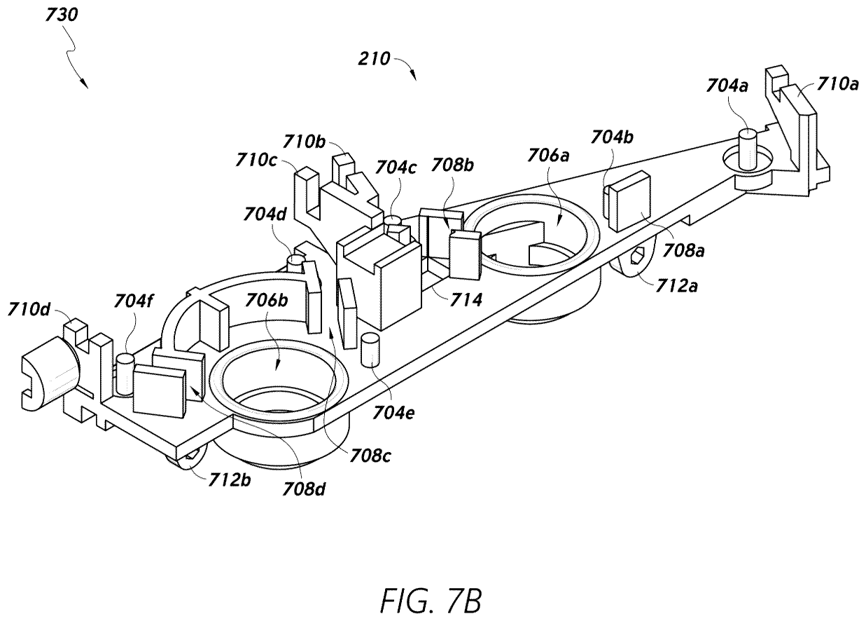

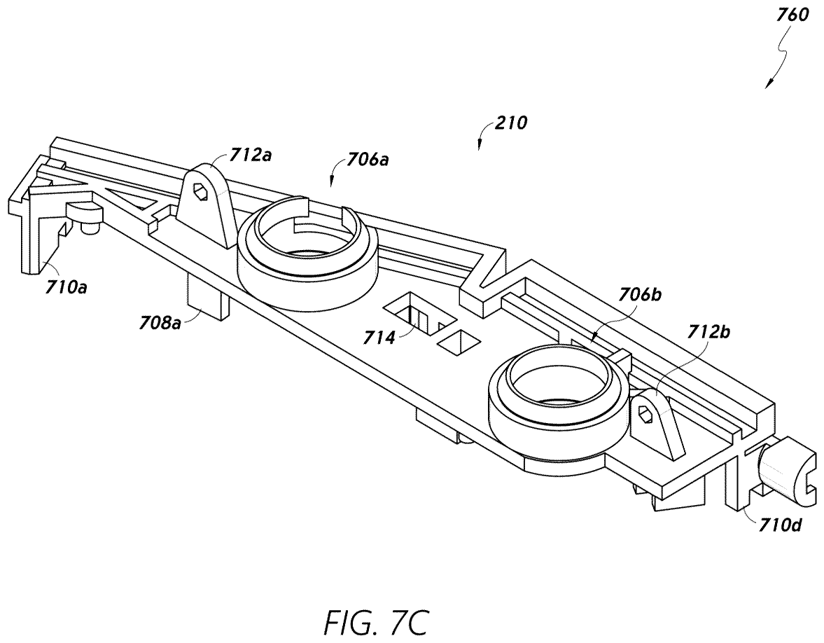

FIGS. 7A-7C illustrate various views 700, 730, 760 of an example chassis 210, which will provide visual references for the following descriptions. The chassis 210 can be configured as a platform for attachment and stabilization of other components. As a platform for attachment, FIGS. 7A-7B show one or more press pins 704a-f on the chassis 210 configured for coupling with their counterparts (e.g., holes or receiving cylinders) on a retainer. It is noted that the press pins 704a-f are exemplary and other fasteners or couplers are possible. Furthermore, it is possible that, instead of the press pins 704a-f shown, their counterpart components may be provided on the chassis 210 with press pins on the retainer.

In some examples, the chassis 210 as the platform can stabilize itself in relation to the casings 502, 504 in FIG. 5 by threading casing rods 512a, 512b into respective holes 712a, 712b on the chassis 210 shown in FIGS. 7B-7C. Furthermore, the chassis 210 can stabilize other components in relation to the chassis 210. For example, the chassis 210 can hold a coupled retainer with its recessed slots on walls 710a-d and support the retainer with its raised tabs 708a, 708b, 708c, 708d.

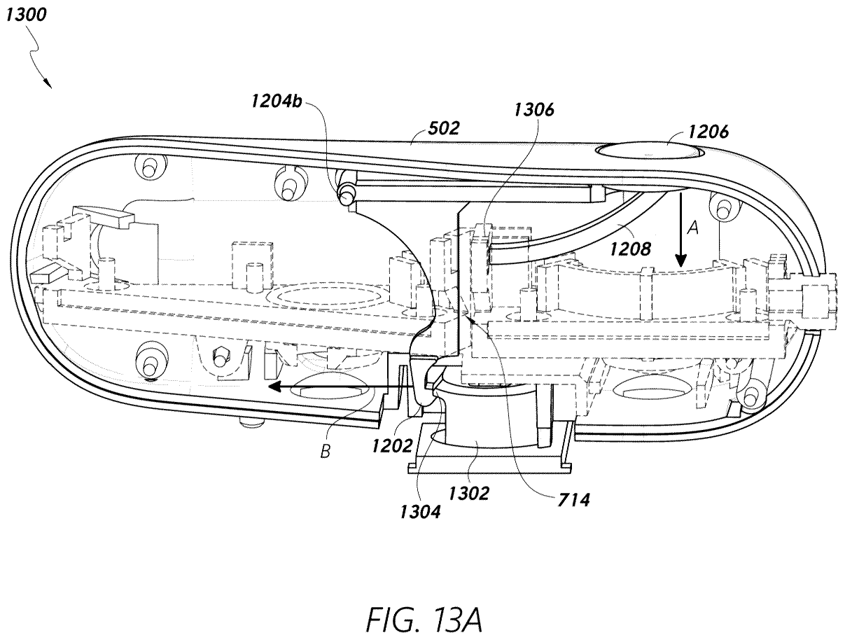

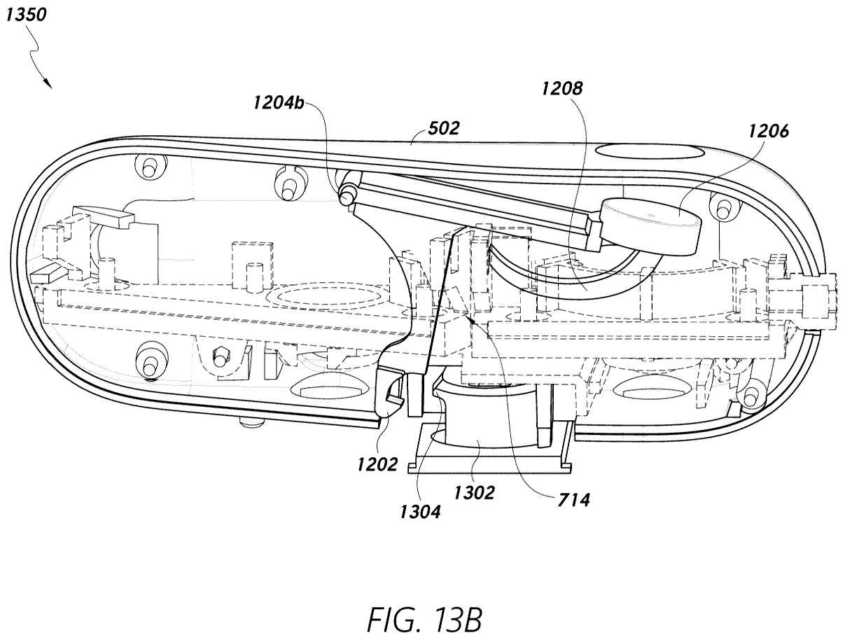

Additionally, the chassis 210 may function as an organizer. As example of an organizer, the chassis 210 can provide one or more spool holders and a latch opening to position and orient each component in association with the chassis 210. The spool holders can be a part of the chassis 210 configured as receptor(s) for rotational components (e.g., the spool(s) 250 of FIG. 5). FIGS. 7A-7C show cylindrical features 706a, 706b as example spool holders. The latch opening can be a part of the chassis 210 configured to pass a hook arm of a latch through and to function as a hard stop in its rotational movement. FIGS. 7B-7C show a cutout 714 configured as the latch opening. The latch opening will be described in greater detail with respect to locked/released configurations shown in FIGS. 13A-13B.

In some examples, the chassis 210 may provide a feature/functionality in connection with another component(s). For example, the chassis 210 can be configured with one or more chassis rail(s) supplied along a side of the chassis 210 that can be one side of a track (e.g., railroad tracks having opposing parallel rails as sides). FIG. 7A illustrates a first chassis rail 702a and a second chassis rail 702b.

In some examples, the chassis 210 may additionally provide walls in connection with its rails 702a, 702b to confine sliding of a slidable component to within a boundary of the track. For example, FIG. 7A shows four walls 710a-d that are found at the ends of each rail 702a, 702b. It is contemplated that such walls may be provided on the retainers instead of, or in addition to, the walls 710a-d on the chassis 210.

### Retainer

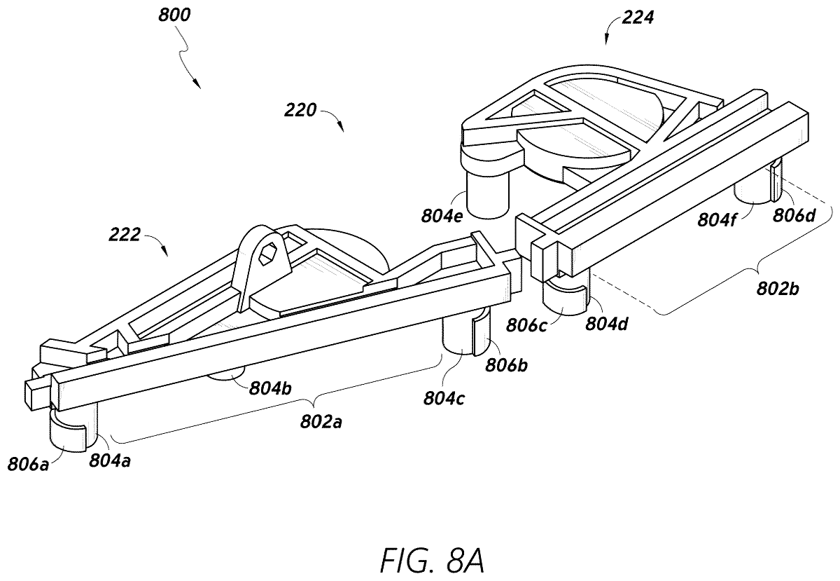

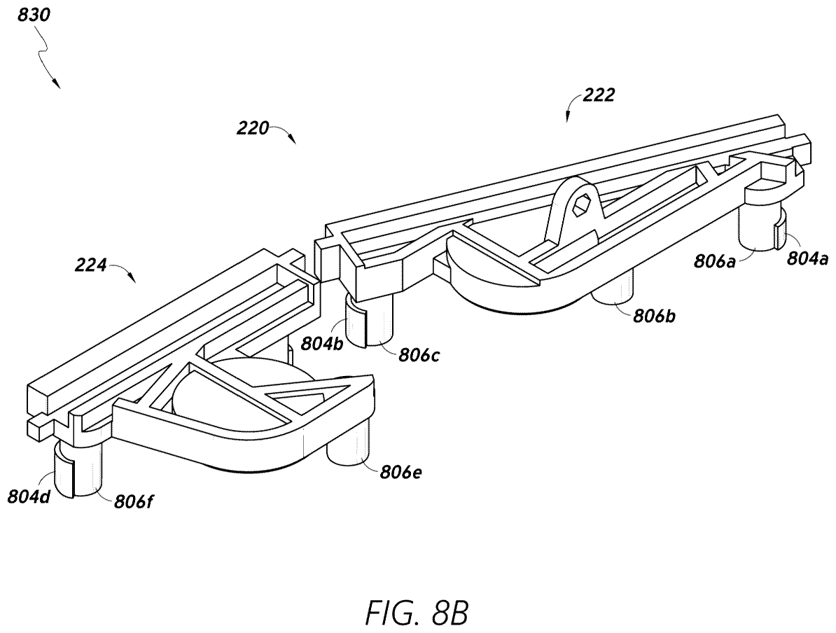

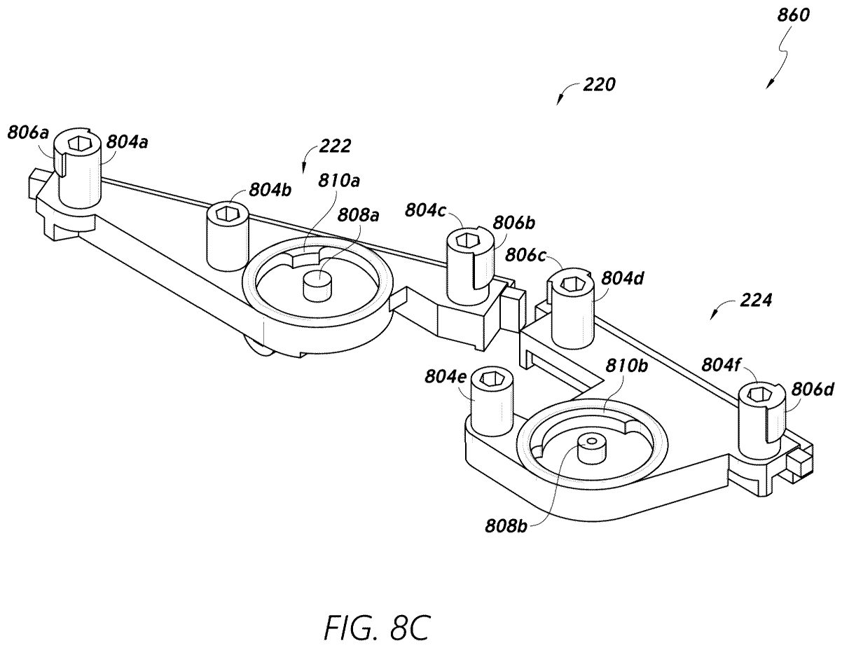

FIGS. 8A-8C illustrate various views 800, 830, 860 of example retainers(s) 220, which will provide visual references for the following descriptions. The retainer(s) 220 can be configured to couple with a chassis (e.g., the chassis 210 of FIG. 5) with one or more receivers 804a-f (e.g., holes for dowel pins) for locking with their counterpart fasteners or couplers (e.g., the press pins 704a-f in FIGS. 7A-7B) on the chassis. As retainer(s) 220, the views 800, 830, 860 illustrate a first retainer 222 and a second retainer 224. The retainer(s) 220 can have structural elements that function as, for example, spool guide, hard stop(s), and retainer rail(s).

The retainer(s) 220 can supply retainer rails 802a, 802b as the other side of the chassis rails 702a, 702b of the chassis 210 in FIG. 7A. For example, a first track can be formed by the first chassis rail 702a of FIG. 7A and an opposing first retainer rail 802a of FIG. 8A and a second track can be formed by a second chassis rail 702b of FIG. 7A and an opposing second retainer rail 802b of FIG. 8A. The first and second tracks can provide linear alignment for slidable components.

The spool guide can be configured to guide rotation of a spool and path strings (e.g., pull wires or tendons) that convert spool rotations into translation (e.g., extension/retraction) of an attached working channel tool or a part thereof. A string can be pathed around one or more guide posts (e.g., guide pin, guide pillar, etc.) to change directions of tensions applied on the string. Advantageously, the receivers 804a-f may, in addition to providing the locking mechanism for coupling with their counterpart press pins, serve as the guide posts. For example, a first set of receivers 804a, 804b can additionally serve to guide pathing of a first pull string wound around the first spool and a second set of receivers 804c, 804d can serve to guide pathing of a second pull string wound around the second spool. In some examples, the guide posts can additionally provide lips 806a-d thereon to prevent the string from derailing the paths provided by the guide posts.

As shown in FIG. 8C, the retainer(s) 220 can include axle-like elements 808 on which a core of a spool can be rotatably coupled. For example, a core of a first spool can be rotatably coupled to a first axle element 808a and a core of a second spool can be rotatably coupled to a second axle element 808b.

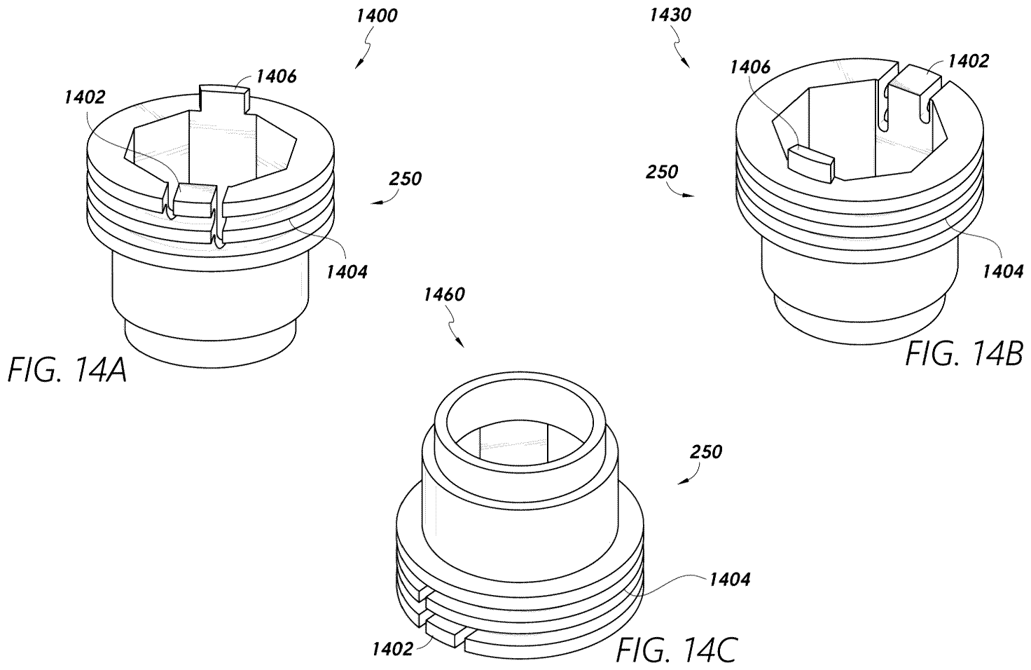

The hard stop(s) can be configured to provide a defined end to spool rotation. The retainer(s) 220 can provide stopper/blocker elements that can prevent further rotation of a spool. While the stopper/blocker elements can take various forms including stopper pins, interlocking tabs, notches, etc., the example retainer(s) 220 in FIG. 8C shows the stopper/blocker elements as bumps 810. Referring to FIGS. 14A-14B, spool(s) 250 can include a stop lip 1406 that, after some rotation, would bump into the bumps 810 that would prevent further spool rotation. For a given size spool, a range of available rotation may be determined based on the lengths of the bumps 810. For example, a smaller bump 810a on a first retainer 222 may enable greater range of rotation for the spool than a longer bump 810b on a second retainer 224.

### Shuttles

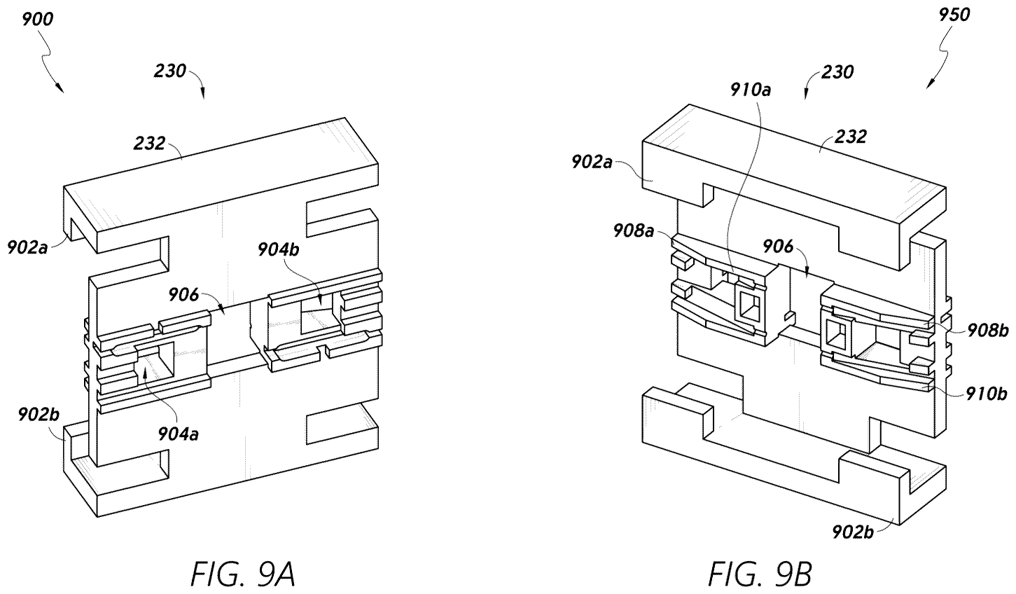

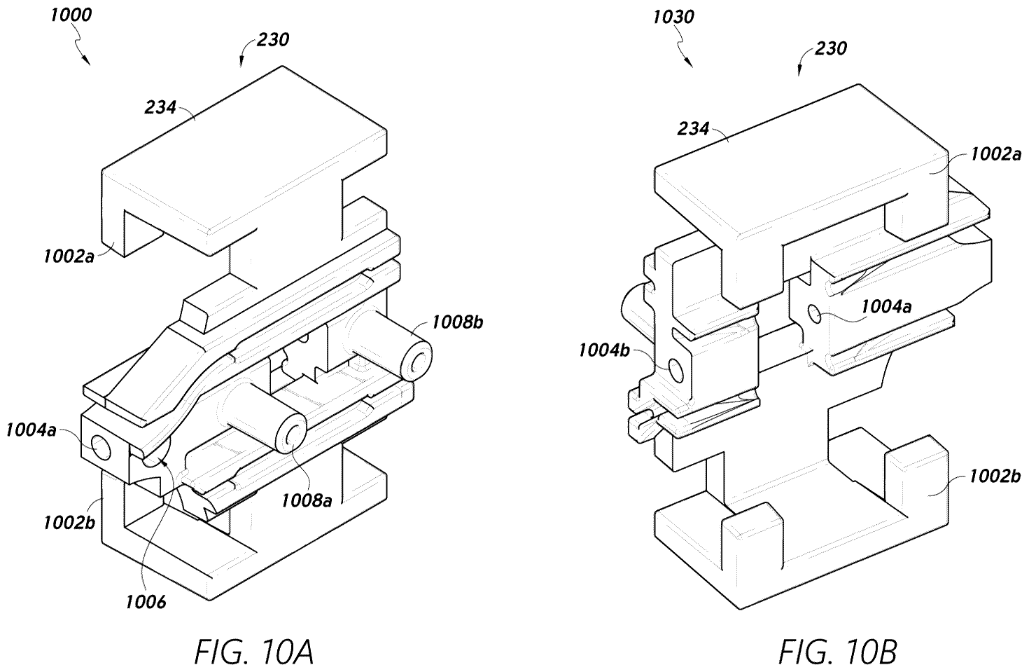

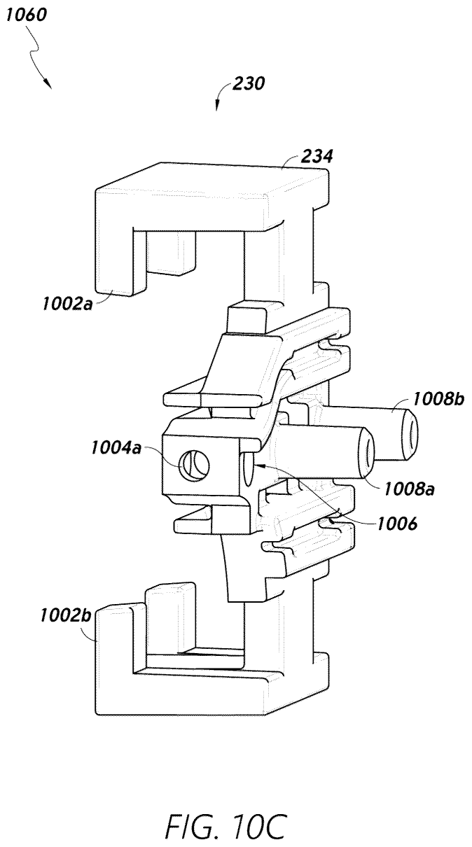

The shuttle(s) 230 can include a first shuttle 232 and a second shuttle 234. FIGS. 9A-9B illustrate various views 900, 950 of the first shuttle 232, which will provide visual references for the following descriptions. The first shuttle 232 can be slidably coupled with the first track previously described. FIGS. 10A-10C illustrate various views 1000, 1030, 1060 of the second shuttle 234, which will provide visual references for the following descriptions. The second shuttle 234 can be slidably coupled with the second track previously described.

As reference, FIG. 11 illustrates an example assembly 1100 of the chassis 210, retainers 220, and shuttles 232, 234, depicting the first shuttle 232 slidably coupled to the first track (denoted ‘A’) and the second shuttle 234 slidably coupled to the second track (denoted ‘B’). The shuttle(s) 230 can have structural elements that function as, for example, hooked edges, bearing(s), string guide, adhesive site, and an attachment interface.

The hooked edges can be configured to slidably lock a shuttle to a track. In some examples, the hooked edges can have a pair of hooks that may reach over and hug both opposing rails of a track. The hooked edges can prevent a shuttle from derailing from the track. The first shuttle 232 hooks onto the first track with the first set of hooks 902a, 902b and the second shuttle 234 hooks onto the second track with the second set of hooks 1002a, 1002b.

The bearing can be configured to provide smooth and controlled sliding of a shuttle on a track. The bearing may, together with the hooked edges, confines movements of the shuttle to the linear movements. In some examples, the bearing may prevent the shuttle from falling into the track.

FIG. 9B shows the bearings as a set of ramps 908a, 908b and a set of bearing rails 910a, 910b. The ramps 908a, 908b can provide smooth translation of the first shuttle 232 on a track. The bearing rails 910a, 910b can, as a form factor, reduce sliding friction between a shuttle and the track.

The string guide can be configured to provide string routing. Referring to FIGS. 10A-10C and its views 1000, 1030, 1060 of the second shuttle 234, the string guide can include guide well(s) 1004a, 1004b that can assist aligning a string (e.g., a pull wire) with the second shuttle 234 by threading the string through the guide well(s) 1004a, 1004b. For example, FIG. 11 shows a string threaded through the guide well(s) of the second shuttle 234.

The adhesive site can be a space on a shuttle to which an adhesive can be applied to fix the string to the shuttle. As an adhesive, a variety of glues (e.g., acrylate, epoxy resin, polyurethane, cement, silicone, etc.) or other adhesives can be applied.

In FIG. 10A and FIG. 10C, the second shuttle 234 shows a glue well 1006 as an example adhesive site. The glue well 1006 may be formed as an internal space on the second shuttle 234 that reveals the string threaded through the guide well(s) 1004a, 1004b. The glue well 1006 facilitates application of glue and prevents the applied glue from flowing over onto other portions of the second shuttle 234.

The attachment interface of a shuttle can facilitate mounting of external component to the shuttle. The shuttle may be, in relation to the casing 504 of FIG. 5, positioned internal to the casing 504 whereas the interfaces 410, 420 of FIG. 4 are positioned external to the casing 504. Accordingly, it can be challenging to provide a manually operable interface (e.g., a grabbable interface such as the first interface 410 or a thumb-slidable interface such as the second interface 420) external to the casing 504.

The attachment interface can facilitate mounting such interfaces for slidably operating a shuttle on a track. For example, the first shuttle 232 slidable on the first track can mount the first interface 410 and the second shuttle 234 slidable on the second track can mount the second interface 420. As shown, the attachment interface can be part of a snap fit coupling (e.g., snap fit receivers 904a, 904b, 906 in FIGS. 9A-9B), a press-pin coupling (e.g., press-pin receivers 1008a, 1008b in FIGS. 10A and 10C), or any other fasteners or connectors.

It will be noted that while some aspects of the bearing, string guide, adhesive site, and attachment interface are described in relation to one of the first shuttle 232 or the second shuttle 234, those features can be provided by any shuttle 230, including the other of the first shuttle 232 or the second shuttle 234.

### Latch

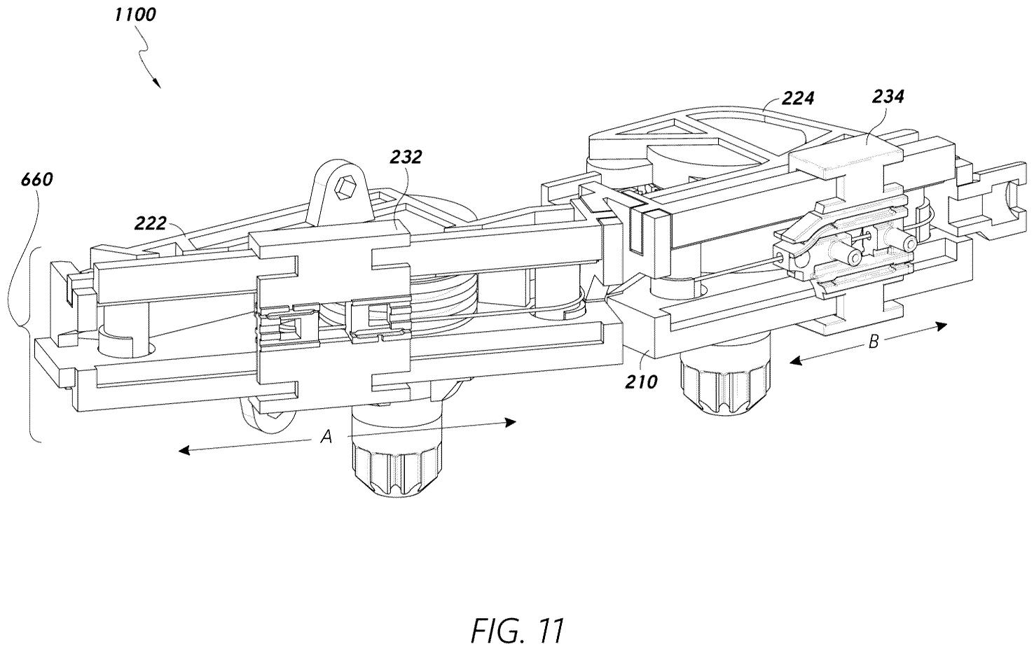

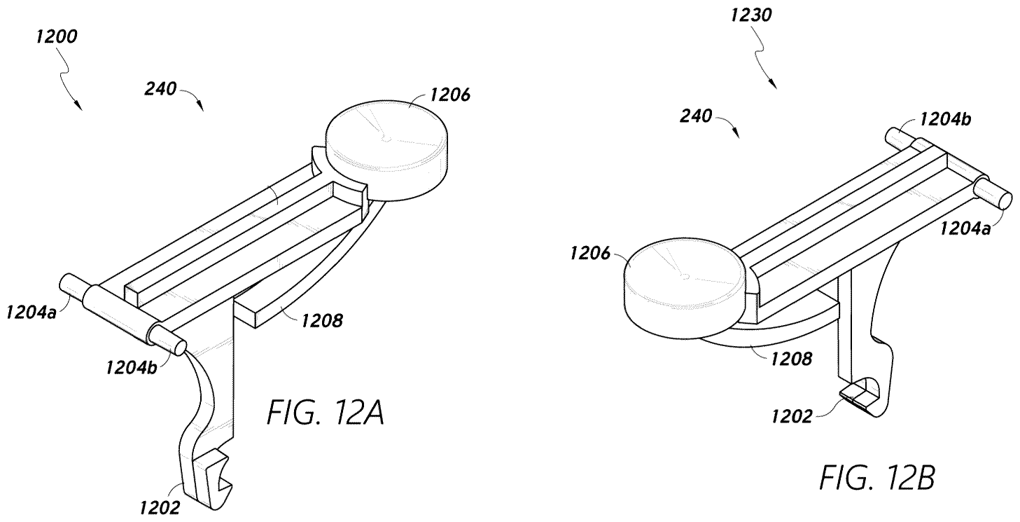

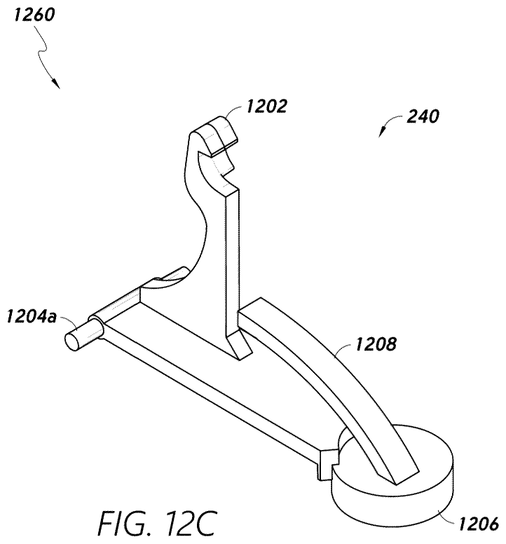

FIGS. 12A-12C illustrate various views 1200, 1230, 1260 of an exemplary latch 240, which will provide visual references for the following descriptions. Broadly, the latch 240 can be a locking mechanism that secures the working channel tool assembly 32 to another component (e.g., the end effector 22 and/or the instrument handle 31 of FIG. 3) to prevent unintended movement or decoupling of the working channel tool assembly 32 therefrom. The exemplary latch 240 can include a hook 1202, a hinge 1204, a user interface button 1206, and an elastic arc 1208.

FIGS. 13A-13B illustrating the latch 240 in, respectively, a locked configuration 1300 and a released configuration 1350 will provide visual references for operations of the latch 240. The latch 240 can have structural elements that function as, for example, a biasing element, a release interface, a locking interface, and a movement ranger.

The biasing element can be a part of the locking mechanism that biases the locking interface toward maintaining the locked configuration 1300 of FIG. 13A. While the biasing element can take various forms, the latch 240 shows the elastic arc 1208, which could be a molded plastic, that functions as a loaded spring. The elastic arc 1208 as the biasing element, when pressure is applied in the direction ‘A’ to the locked configuration 1300 in FIG. 13A, bends or deforms (changes its curvature) as shown in the released configuration 1350 of FIG. 13B since the elastic arc 1208 cannot release the applied pressure through a wall 1306. When the pressure in the direction ‘A’ is released, the elastic arc 1208 can return to its original shape in the locked configuration 1300. In some examples, the chassis 210 may provide a hard stop for the elastic arc 1208 such that the elastic arc 1208 cannot bend (change curvature) beyond a certain curvature.

The release interface can be a part of the locking mechanism that allows release of the working channel tool assembly 32 from an attached component. While the release interface can take various forms, the latch 240 shows the user interface button 1206 as the release interface. In the locked configuration 1300 of FIG. 13A, the user interface button 1206 is pushed up by the elastic arc 1208 against an interface cutout (e.g., the interface cutout 508 of FIG. 5) such that the user interface button 1206 is pressable in the direction ‘A’. In the released configuration 1350 of FIG. 13B, the user interface button 1206 is pressed in the direction ‘A’ causing the elastic arc 1208 to bend or deform. A hinge 1204 translates the press in the direction ‘A’ to movement of the hook 1202 in the direction ‘B’, and move the hook 1202 away from a lip 1304 of a coupling interface 1302 of the attached component. The released configuration 1350 allows decoupling of the working channel tool assembly 32 from the coupling interface 1302.

The locking interface can be a part of the locking mechanism that holds the working channel tool assembly 32 to the attached component. While the locking interface may take various forms, the latch 240 shows the hook 1202 as the locking interface. In the locked configuration 1300 of FIG. 13A, the hook 1202 is tightly pushed against a lip 1304 of the coupling interface 1302, locks with the lip 1304, and prevents separation between the working channel tool assembly 32 and the instrument base 31. In the released configuration 1350 of FIG. 13B, the hook 1202 is pulled away from the lip 1304 of the coupling interface 1302, unlocks with the lip 1304, and allows separation between the working channel tool assembly 32 and the attached component.

The movement ranger can be a part of the locking mechanism that limits range and/or controls direction of movement of the locking interface. While the movement ranger can take various forms, the latch 240 shows the hinge 1204 as the movement ranger. As shown in FIG. 12A-12B, the hinge 1204 can have a first hinge pin end 1204a and a second hinge pin end 1204b on opposing sides that can be pressed into and held by their counterparts on the casings 502, 504 of FIG. 5. The counterparts of the casings 502, 504 can loosely hold the first hinge pin end 1204a and the second hinge pin end 1204b so to allow rotational movement of the locking interface about the counterparts. The locked configuration 1300 of FIG. 13A and the released configuration 1350 of FIG. 13B show the rotational movement.

Additionally, as described, the chassis 210 may provide a latch opening (shown as a cutout 714 in FIGS. 7B-7C) configured to pass a hook arm of a latch 240 through and to function as a hard stop limiting range of rotation. As shown in FIGS. 13A-13B, the cutout 714 can allow limited movement of the hook 1202 toward and away from the lip 1304.

### Spools

FIGS. 14A-14C illustrate various views 1400, 1430, 1460 of the spool(s) 250, which will provide visual references for the following descriptions. Here, the spool(s) 250 may refer to either or both of a first spool 252 or a second spool 254, each configured to wind or unwind a string (e.g., a pull wire) fixedly attached to the spool. When the working channel tool assembly 32 is configured with a basket tool 33 of FIG. 2B, a first drive output coupled to the first spool 252 can be configured to control extension/retraction of the basket sheath 34 and a second drive output coupled to the second spool 254 can be configured to control open/close of the basket 35 through controlling extension/retraction of the wire tines 36. In some examples, the spool(s) 250 can provide a smooth bearing surface for the string. The spool(s) 250 can have structural elements that function as, for example, a capstan and a stop feature.

The capstan can be a part of a spool that serve as a fixation point (termination point) for a string (e.g., a pull wire) to be wound around the spool on a bearing surface 1404. As shown in FIGS. 14A-14C, the spool has a capstan feature 1402. Instead of gluing an end of the string to the fixation point to couple the spool with the string, the capstan feature 1402 can be used as a post for wrapping/tying the string thereby securing it sufficiently under applied tensions. Advantageously, the capstan feature 1402 removes the use of adhesives and curing time while smoothening the bearing surface 1404.

The stop feature can be a part of a spool that causes rotation of the spool 250 to be limited in range. As shown in FIGS. 14A-14C, the spool can have a stop lip 1406. When the spool is coupled with and rotated about the axle elements 808 of the retainer(s) 220 shown in FIG. 8C, the stop lip 1406 can be stopped by the bump 810 of the retainer(s) 220.

### Working Channel Tool Assembly Process

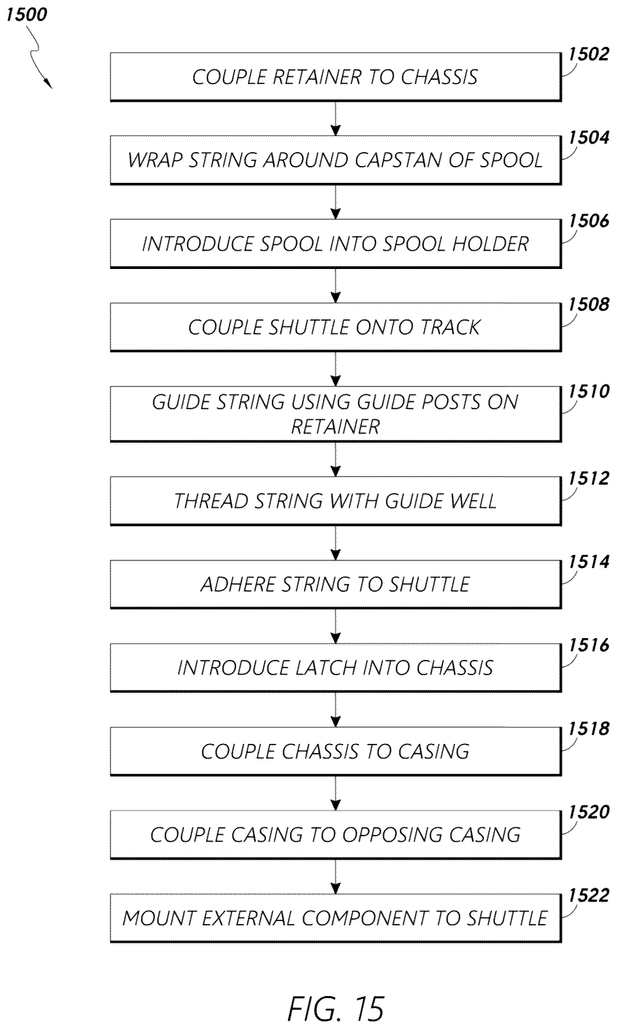

FIG. 15 illustrates a flow diagram for an example process 1500 for assembling a working channel tool assembly, in accordance with some implementations. The process 1500 may include additional blocks or fewer blocks and, in some examples, blocks out of order compared to the order shown.

At block 1502, the process 1500 involves coupling a retainer (e.g., the retainer(s) 220 of FIG. 5) to a chassis (e.g., the chassis 210 of FIG. 5). In some examples, the retainer has thereon a rail-like structure and the chassis has another rail-like structure that, when the retainer is coupled to the chassis, form two opposing rails of a track. For example, when the chassis 210 is coupled to the first retainer 222, a chassis rail 702a and a retainer rail 802a can form the opposing rails of the track.

At block 1504, the process 1500 involves wrapping or tying a string around a capstan (e.g., the capstan feature 1402 of the spool(s) 250 shown in FIGS. 14A-14C) of a spool.

At block 1506, the process 1500 involves introducing the spool into a spool holder (e.g., the cylindrical features 506a, 506b shown in FIGS. 7A-7C) on the chassis and configuring the spool to rotate about an axle (e.g., axle elements 808a, 808b of the respective retainer(s) 222, 224 shown in FIG. 8C) on the retainer.

At block 1508, the process 1500 involves coupling a shuttle (e.g., the shuttle 230 of FIG. 5) onto the track formed by the coupling of the retainer to the chassis, a first side of the track formed on the chassis and a second side of the track formed on the retainer.

At block 1510, the process 1500 involves guiding the string of the spool using one or more guide posts formed on the retainer. In some examples, cylinders (e.g., any of the receivers 804a-d in FIGS. 8A-8C) usable for coupling between the retainer and the chassis at block 1502 can be configured serve as the guide posts. For example, the string can be redirected using the cylinders to guide pathing of the string.

At block 1512, the process 1500 involves threading the string with a guide well on the shuttle. In some examples, the string can be introduced into the guide well (e.g., the guide wells 1004a, 1004b of the second shuttle 234 shown in FIGS. 10A-10C).

At block 1514, the process 1500 involves gluing a portion of the string to the shuttle at a glue well (e.g., the glue well 1006 of the second shuttle 234 shown in FIGS. 10A and 10C) on a surface of the shuttle.

At block 1516, the process 1500 involves introducing a locking interface of a latch through an opening formed on the chassis of the first assembly. In some examples, the locking interface may be a hook (e.g., the hook 1202 of FIGS. 12A-12C) introduced into the cutout 714 of the chassis 210 in FIGS. 7B-7C). As described, the opening may further function as a hard stop for movements of the latch.

At block 1518, the process 1500 involves coupling the chassis and a first casing. In some examples, the coupling can involve threading rods (e.g., rods 512a, 512b on the first casing 502 of FIG. 5) into respective holes (e.g., 712a, 712b on the chassis 210 shown in FIG. 7C) such that the chassis is stabilized on the first casing. During the coupling, a first end of a hinge pin (e.g., the first hinge pin end 1204a of FIGS. 12A-12C) of the latch can be inserted into a first hinge pin receptor formed on the first casing.

At block 1520, the process 1500 involves coupling the first casing to a second casing opposing the first casing. The first casing and the second casing may be opposing halves of a casing. In some examples, the coupling can involve mating press pins (e.g., press pins 514a, 514b on the first casing 502 of FIG. 5) and counterpart cylinders (e.g., receptors described in relation to the second casing 504 of FIG. 5) on the second casing to enclose all of the assembled components inside.

During the coupling, a second end of a hinge pin (e.g., the second hinge pin end 1204b of FIGS. 12A-12B) of the latch can be inserted into a second hinge pin receptor formed on the second casing to rotatably fix the latch. When both ends of the hinge pin is held by the first and second casings, the latch can be rotatably fixed at the hinge.

At block 1522, the process 1500 involves mounting an external component to an attachment interface of the shuttle. In some examples, the external component mounted can be an interface to facilitate sliding of the shuttle on the track.

### Additional Embodiments

Depending on the embodiment, certain acts, events, or functions of any of the processes or algorithms described herein can be performed in a different sequence, may be added, merged, or left out altogether. In addition, various features of different disclosed embodiments can be combined to form additional embodiments, which are part of this disclosure. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

1. A working channel tool assembly, comprising:
a chassis providing a first side rail and a first assembly interface;
a retainer providing a second side rail and a second assembly interface, the second assembly interface being configured to mate with the first assembly interface; and
a first track formed by the first and second side rails when the chassis couples with the retainer mating the first assembly interface to the second assembly interface.

2. The assembly of claim 1, wherein the chassis and the retainer are each made of a single molded plastic.

3. The assembly of claim 1, further comprising a first shuttle having at least two opposing hooked edges operable to slidably couple the first shuttle to the first track.

4. The assembly of claim 3, wherein the shuttle is fixedly coupled with a portion of a tool for insertion into a lumen of an endoscope, and wherein sliding the first shuttle advances or retracts the tool within the lumen.

5. The assembly of claim 4, wherein the tool is at least one of a laser tool or a basket tool.

6. The assembly of claim 3, further comprising a casing to internally house the chassis, the retainer, and the first shuttle, wherein the first shuttle has an attachment interface configured to mount an external component through a slot formed on the casing, the slot extending parallel the track.

7. The assembly of claim 3, wherein the first shuttle defines a guide well through which a string is threadable.

8. The assembly of claim 7, wherein the first shuttle defines a glue well that exposes a portion of a string, wherein glue is receivable within the glue well to adhere the string to the first shuttle.

9. The assembly of claim 3, further comprising:
a second track jointly formed by a third side rail provided by the chassis and a fourth side rail provided by the retainer; and
a second shuttle slidably coupled to the second track.

10. The assembly of claim 9, wherein the first shuttle is fixedly coupled with a first string operable to control insertion and retraction of a first part of the tool, and the second shuttle is fixedly coupled with a second string operable to control insertion and retraction of a second part of the tool.

11. The assembly of claim 10, wherein the first part of the tool is a basket sheath of a basket tool, and the second part of the tool is at least one wire configured to telescopically slide within and in relation to the basket sheath.

12. The assembly of claim 1, wherein the chassis comprises walls at opposing ends of the first side rail, the walls being configured to limit movement range of a component coupled to the first side rail.

13. The assembly of claim 1, wherein the chassis comprises a latch opening through which a locking mechanism of a latch is extendable.

14. The assembly of claim 1, wherein the second assembly interface is a guide post configured to guide a string.

15. The assembly of claim 1, wherein the retainer includes an axle sized to receive a spool for rotation thereon, and a stopper to limit rotation of the spool.

16. The assembly of claim 1, further comprising a spool including a capstan feature configured to fix an end of a string wound around the spool to the spool.

17. A working channel tool assembly, comprising:
a first casing providing a first assembly interface;
a second casing providing a second assembly interface, the first casing being coupled to the second casing by mating the first assembly interface to the second assembly interface;
a latch comprising:
a hinge;
a biasing element; and
a locking interface; and
at least one component configured to control insertion or retraction of a tool within a working channel of an elongate shaft coupled to a base,
wherein mating the first and second casings limits a range of movement for the latch to rotate about the hinge, and
wherein the biasing element urges the latch to a locking configuration when the locking interface engages with a portion of the base.

18. The assembly of claim 17, further comprising a release interface manually engageable to deform the biasing element and thereby rotate the latch to a release configuration where the locking interface disengages from the portion of the base.

19. The assembly of claim 17, wherein the biasing element is made of molded plastic having elastic property and the locking interface is a hook.

20. The assembly of claim 17, wherein the biasing element is a deformable arc that changes curvature.

21. A shuttle comprising:
a pair of hooked edges configured to hook onto opposing rails of a track, the hooked edges formed on a first side of a molded plastic;
a string guide configured to provide string routing;
an adhesive site to which an adhesive can be applied to fix the string to the shuttle; and
an attachment interface configured to mount an external component,
wherein the shuttle is housed within a first side of a casing, and wherein the attachment interface is configured to mount the external component on a second side of the casing opposing the first side.
